(19) United States
(12) Patent Application Publication
(10) Pub. No.: US 2010/0036368 A1
(43) Pub. Date: Feb. 11, 2010

(54) Method of selectively heating adipose tissue

(76) Inventors: Laura England, Alameda, CA (US); Dragan Nebrigic, Carlsbad, CA (US)

Correspondence Address: WOOD , HERRON & EVANS, LLP (SOLTA), 441 VINE STREET, 2700 CAREW TOWER, CINCINNATI, OH, 45202, US

(21) Appl. No.: 12/462,863

(22) Filed: Aug. 11, 2009

Related U.S. Application Data
(60) Provisional application No. 61/087,874, filed on Aug. 11, 2008.

(51) Int. Cl. A61B 18/14; A61B 18/18

(52) U.S. Cl. 606/33; 606/41

(57) ABSTRACT

A method of operating a tissue treatment apparatus for heating adipose tissue located beneath the dermis with high frequency energy from an electrode. The method includes delivering high frequency energy from the electrode to the adipose tissue at a dose that is applied over a period of time sufficient to heat the adipose tissue while at the same time not cause significant heating of the dermis. Embodiments of the method include cooling the electrode to cool a region of tissue next to the electrode.

### CROSS REFERENCE TO RELATED APPLICATIONS

This application claims the benefit of U.S. Provisional Application Ser. No. 61/087,874, filed on Aug. 11, 2008, the disclosure of which is expressly incorporated by reference herein in its entirety.

### BACKGROUND

The invention generally relates to methods for treating tissue with high frequency energy and, more particularly, relates to methods for delivering the high frequency energy to adipose tissue.

Radiofrequency (“RF”) energy has the ability to deeply penetrate tissues for performing therapeutic tissue treatments. This ability, combined with the distinctive bioelectric properties of fat, as well as the anatomy of adipose tissue, make RF energy well suited for use in noninvasive adipose tissue reduction therapies. Thermal applications or traumas are well known to cause apoptosis and necrosis in adipose tissues which result in fat cell elimination. The major component of adipose tissue is fat. Fat has a lower specific heat than dermal collagen. Therefore, less energy is required to raise the temperature of fat containing adipose tissue as compared to dermal collagen. Fat also has a lower dielectric permittivity and higher thermal coefficient which means that, although it takes longer to heat adipose tissues such as subcutaneous adipose tissue, the fatty tissue retains heat for a longer period of time compared to non-fatty tissues. In addition, blood perfusion in subcutaneous adipose tissue is relatively low compared to nearby dermal tissue. The relatively low perfusion of blood in subcutaneous adipose tissue favors heating while the relatively high blood perfusion of the dermal tissue aids cooling. Differences between adipose tissue and dermal tissue are illustrated in Table 1 below.

TABLE 1

| | Dermis | Adipose |
|---|---|---|
| Specific heat: | 3.8 × 103 J/kg/°K | 2.3 × 103 J/kg/°K |
| Blood perfusion: | 120 ml/kg/min | 28 ml/kg/min |
| Dielectric permittivity | 600 | 20 |
| (frequency dependent): | | |

RF energy may be delivered to tissue by monopolar treatment tips. Commercialized monopolar treatment tips apply RF energy to tissues while simultaneously cooling the surface of the skin in treatment cycles. For example, current monopolar treatment cycles include approximately 5 to 200 milliseconds of pre-cooling, approximately 1 to 1.5 seconds of RF energy application time, and a post cool period which varies from 500 milliseconds to 800 milliseconds depending on the treatment tip target heating depth and the application. The short RF energy application times used with current devices and methods, i.e., 1 to 1.5 seconds, favors heating of collagen containing structures, namely the dermis and subcutaneous fibrous septae, because of the high conductance of the collagenous structures.

### SUMMARY

Embodiments of the invention relate to a method of operating a tissue treatment apparatus for heating adipose tissue located beneath the dermis. In one embodiment, the method includes delivering the high frequency energy from an electrode to the adipose tissue at a dose and for a period of time that are sufficient to heat the adipose tissue to at least 42° C. with the proviso that the temperature of the dermis is about 42° C. or less and the dose is in a range of about 3 W/cm² to about 100 W/cm², and the period of time ranges from greater than 3 seconds to about 30 seconds. In another embodiment, the method includes delivering high frequency energy from an electrode to the adipose tissue at a dose and for a period of time that are sufficient to increase at least one of necrosis, apoptosis, or inflammation in the treated tissue relative to an untreated tissue and the dose is in a range of about 3 W/cm² to about 100 W/cm², and the period of time ranges from greater than 3 seconds to about 30 seconds. In yet another embodiment, the methods includes delivering high frequency energy from an electrode to the adipose tissue at a dose and for a period of time that are sufficient to decrease perilipin activity and the dose is in a range of about 3 W/cm² to about 100 W/cm², and the period of time ranges from greater than 3 seconds to about 30 seconds.

### BRIEF DESCRIPTION OF THE DRAWINGS

This patent or application file contains at least one drawing executed in color. Copies of this patent or patent application publication with color drawing(s) will be provided by the Office upon request and payment of the necessary fee.

The accompanying drawings, which are incorporated in and constitute a part of this specification, illustrate embodiments of the invention and, together with a general description of the invention given above, and the detailed description given below, serve to explain the principles of the embodiments of the invention.

FIG. 1 is a graph illustrating energy delivered over time in accordance with an embodiment of the invention.

FIG. 2A is a graph illustrating a computer model of an embodiment of the invention.

FIG. 2B is a graph illustrating a computer model of an embodiment of the invention.

FIG. 3A is a graph illustrating a computer model of an embodiment of the invention.

FIG. 3B is a graph illustrating a computer model of an embodiment of the invention.

FIG. 4 is a graph illustrating the selective heating of fat with an embodiment of the invention.

FIG. 5 is a graph illustrating the stepped heating of fat with an embodiment of the invention.

FIG. 6 is photomicrograph of a histological section showing the effect of an embodiment of the invention on tissue 24 hours after treatment.

FIG. 7 are photomicrographs of histological sections showing the effect of an embodiment of the invention on tissue 2, 4, 6, and 8 weeks after treatment.

FIG. 8 are photomicrographs of histological sections showing the effect of embodiments of the invention on tissue.

FIG. 9 is a graph illustrating decrease in fat cell numbers in a tissue as a result of treatment with an embodiment of the invention.

FIG. 10 are photomicrographs of histological sections demonstrating changes in perilipin expression and macrophage infiltration that results from treatment of tissue using embodiments of the invention.

### DETAILED DESCRIPTION

New treatment protocols for delivering RF energy to cause the selective heating of tissue having a relatively high fat content, such as adipose tissue, are described herein. The treatment protocols utilize a method of delivering RF energy to a tissue while concurrently cooling the surface of the tissue. Devices that may be used with this improved methodology have been previously described in U.S. Publication No. 2009/0018628, and devices described in Provisional Application Nos. 61/226,138, 61/226,140, and 61/226,145, which are hereby incorporated by reference herein in their entirety. The devices may be monopolar or bipolar, and may require some modification for optimum performance with these new treatment protocols, such as increasing the tip surface area or redesigning the heat spreader, modifying the electrode geometry, and altering the RF power or current profiles during delivery to achieve uniform tissue heating and predictable homogenous fat reduction.

The new treatment protocols include the selective heating of fat containing tissues, such as adipose tissue, an example being subcutaneous adipose tissue, by delivering a relatively low dose of RF energy over a relatively long period of time. As used herein, dose may be used to indicate the RF energy (expressed in joules) or the power (expressed in watts) delivered to an area of 1 cm². The area over which the dose is delivered is not limited to an area of 1 cm², and it is contemplated that the dose may be delivered to larger or smaller areas. The dose, as referred to herein, is the RF energy applied in an individual pulse.

The delivered RF energy may be in a range of about 9 J/cm² to about 800 J/cm². In another embodiment, the delivered RF energy is in a range of about 9 J/cm² to about 400 J/cm². More particularly, the delivered RF energy may be in range of about 12 J/cm² to about 300 J/cm². More particularly, the delivered RF energy may be in range of about 15 J/cm² to about 200 J/cm². More particularly, the delivered RF energy may be in range of about 18 J/cm² to about 100 J/cm². More particularly, the delivered RF energy may be in range of about 24 J/cm² to about 800 J/cm². The RF energy may be delivered over a period of time greater than 3 seconds to about 30 seconds, or about 4 seconds to about 30 seconds, or about 4 seconds to about 24 seconds, or about 6 seconds to about 10 seconds, or about 8 seconds. Generally, higher energy doses are associated with shorter periods of time.

The delivered power of RF energy may be in a range of about 3 W/cm² to about 100 W/cm². In one embodiment, the delivered power is in a range of about 3 W/cm² to about 70 W/cm², and more particularly in a range of about 5 W/cm² to about 17 W/cm², and more particularly in a range about 10 W/cm² to about 14 W/cm². The RF energy may be delivered over a period of time greater than 3 seconds to about 30 seconds, or about 4 seconds to about 30 seconds, or about 4 seconds to about 24 seconds, or about 6 seconds to about 10 seconds, or about 8 seconds. Generally, higher energy doses are associated with shorter periods of time.

RF energy may be applied in a single pulse or a series of pulses. When a series of pulses is desired, the pulses may be delivered using the stepped method wherein the tissue is not allowed to cool down to pre-stimulation temperature levels between pulses, or the tissue may be allowed to cool down to approximately normal core body temperature and or skin surface temperature between pulses.

RF energy should be applied at a dose and rate to heat the fat containing tissue, i.e., adipose tissue with an example being subcutaneous adipose tissue, to at least 42° C., or, more particularly, to 45° C. or higher, or, more particularly, to 50° C. or higher, or, more particularly, to 55° C. or higher. The RF energy dose and rate should be also be applied such that the temperature of the dermis is about 45° C. or less, or, more particularly, about 44° C. or less, or, more particularly, about 43° C. or less, or, more particularly, about 42° C. or less, or, more particularly, about 41° C. or less, or, more particularly, about 40° C. or less, or, more particularly, about 37° C. or less. Temperature of the dermis and/or adipose tissue may be measured by any method known to one having ordinary skill in the art, such as, without limitation, by contact probe or a thermo-imaging.

In accordance with the improved treatment protocols described herein, RF energy is applied to tissue in a dose and at a rate sufficient to induce at least one of necrosis, apoptosis, and inflammation as may be indicated by various histological staining methods such as decreased fat cell count, increased infiltration by macrophages or lymphocytes, increased TUNEL staining and/or caspase activity, increased accumulation of HSP 47, CD 68, and/or collagen deposition, and/or decreased LDH and/or perilipin activity. Some of these parameters may be measured at least 24 hours to about three months after RF energy application.

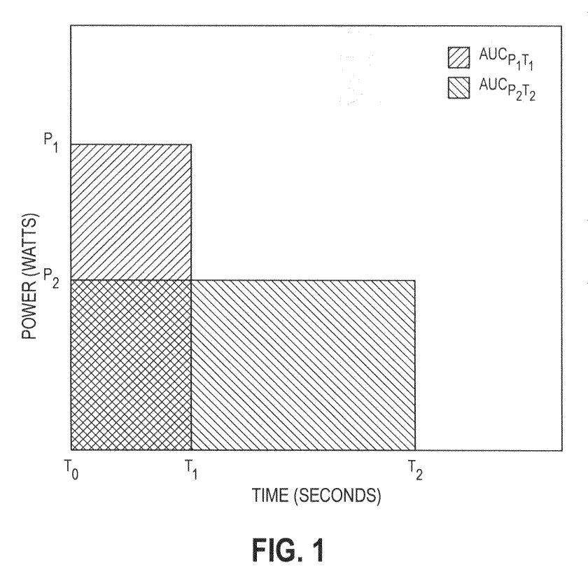

Treatment parameters on a deep heating monopolar treatment tip are modified to allow extended RF energy application times. RF energy application times range from 3 to 30 seconds with concurrent contact cooling. Various rationales support extending the time period for RF energy application. One rationale is that longer RF energy application time may allow the low conductivity fat component of adipose tissue to respond with an increase in temperature. Another rationale is that longer RF energy treatment times may allow thermal damage to occur at lower temperatures (time-temperature dependence of thermal damage). Yet another rationale is the cryogen cooled tip surface has a greater capability to cool the epidermis and dermis at the relatively lower powers described herein and that the application of RF energy at a lower power (J/sec) over an extended period of time increases the total energy (J/cm²) delivered that a patient may tolerate. This concept is illustrated in FIG. 1 wherein P₁ is the power that a patient can tolerate for the time period from T₀ to T₁, (for example, about 150 J/sec delivered over 1 second). The area under the curve (“AUC”) for P₁T₁ is the total energy (for example, about 150 J/cm²) that a patient can tolerate for the time period from T₀ to T₁. P₂ is the power that a patient can tolerate if the energy is delivered over a longer period of time, i.e., T₀ to T₂ (for example, 37.5 J/sec delivered for 8 seconds). The area under the curve for P₂T₂, i.e., the total energy that a patient can tolerate with the extended delivery (for example, about 300 J/cm²), is greater than the area under the curve for P₁T₁ (for example, about 150 J/cm²). Thus, the greater amount of energy delivered over time period T₀ to T₂ is more tolerable to a patient, or at the least, not less tolerable, than the lower amount of energy delivered over time period T₀ to T₁.

The tolerability of an energy treatment is generally related to the resulting peak temperature reached in a tissue. Treatments that generate higher temperatures are typically deemed less tolerable. RF energy is absorbed more quickly by the fibrous septae and skin as compare to fat tissue. As a result, the temperature of fibrous septae and skin increases more quickly than that of fat tissue. However, the fibrous septae and skin have greater thermal conductivity and dissipation of heat by blood flow than fat tissue. The higher power energy delivered during P₁T₁ results in heating of the fibrous septae and skin so quickly that the heat is not dissipated before reaching higher peak temperatures resulting in lower tolerability. In contrast, delivering energy at a lower power over a longer period of time, i.e., P₂T₂, allows the fibrous septae and skin to conduct away and dissipate the heat, thereby increasing the tolerability of the treatment while at the same time allowing sufficient heating of the fat tissue to have a therapeutic effect.

### EXAMPLE

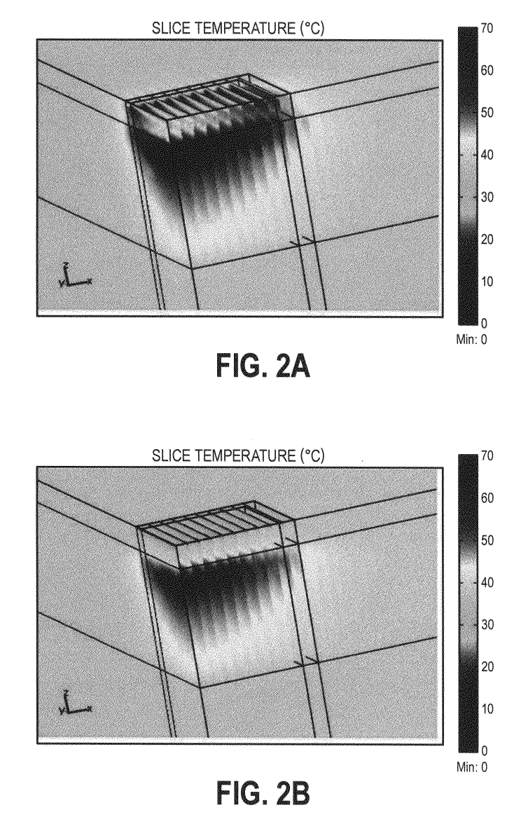

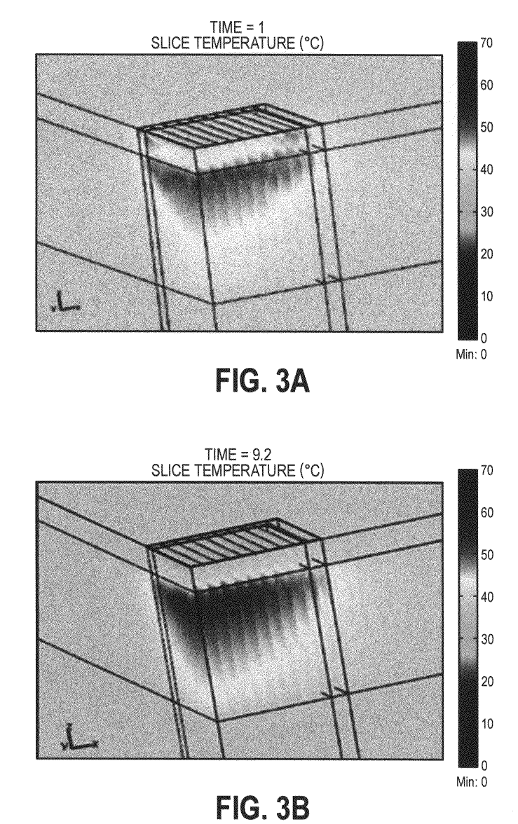

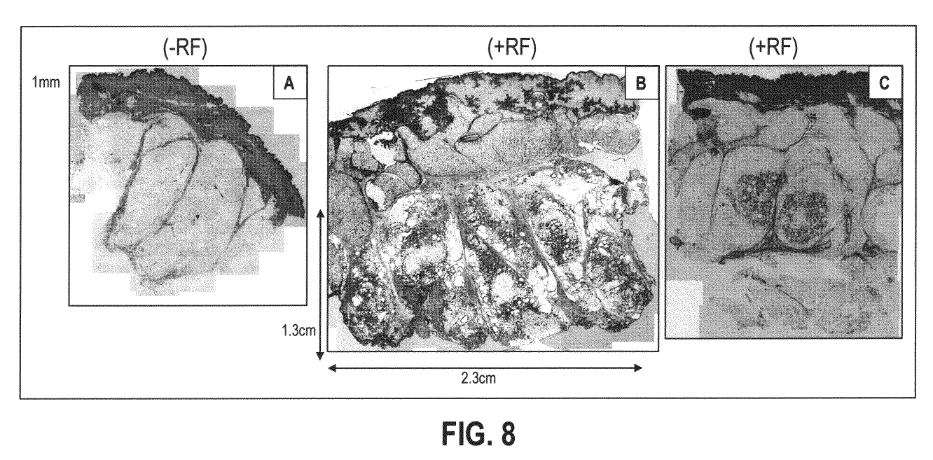

Multi-layer computer models (developed using software commercially available from Comsol®) were constructed that incorporated the bioelectrical properties, typical thicknesses and characteristic blood perfusion rates of the tissue layers (epidermis, dermis, fat, muscle). Such multi-layer computer models and their use are understood by a person having ordinary skill in the art. In FIGS. 2A, 2B, 3A, and 3B, illustrating the results of the multilayer computer modeling, the uppermost layer of the modeled tissue is a 2 mm thick layer of epidermis/dermis. Superficial to the dermal layer is a 10 mm thick layer of adipose tissue, and deep to the adipose tissue is a layer of muscle tissue. The thickness of the fat was based upon the juvenile pig model. Adult human fat may be substantially thicker in certain areas such as thigh or abdomen. FIGS. 2A and 2B illustrate peak heating profiles of simulations of RF energy delivered to an area of 3 cm² for 1 second (FIG. 2A) or 8 seconds (FIG. 2B) in conjunction with contact cooling. The simulations indicated dermal and subdermal heating when a given amount of RF energy was applied for 1 second and selective subdermal heating when the same amount of RF energy was applied over a period of 8 seconds.

One second RF energy applications were compared to 8 second RF energy applications on abdomen skin of humans in order to evaluate relative comfort levels of the two different parameters. An 8 second RF energy application resulted in tolerable comfort of an average of 63% more total energy delivered than if delivered over 1 second. To compare this difference, computer simulations were run at 184 J/3 cm² for 1 second of RF energy and at 300 J/3 cm² for 8 seconds of RF energy. Peak heating profiles of 1 second versus 8 second RF energy applications at equivalent comfort levels are shown in FIGS. 3A and 3B, respectively. The RF energy application of 184 J/3 cm² over 1 second resulted in more dermal heating and less subcutaneous heating. The RF energy application of 300 J/3 cm² over 8 seconds resulted in little or no dermal heating and significant subcutaneous heating.

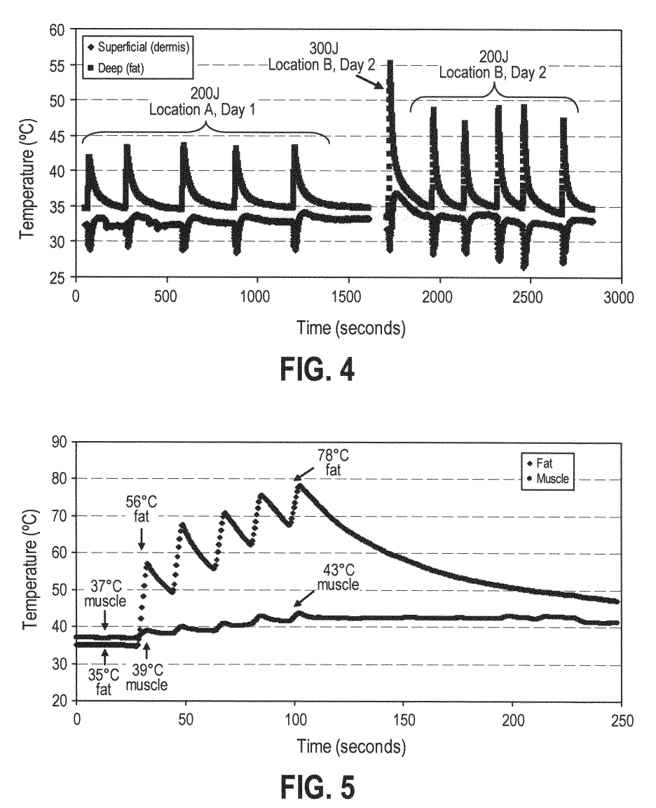

Infrared camera thermal imaging and in vivo temperature measurements were conducted to confirm predictions of the computer simulations. The in vivo temperature measurements were performed using multiple fluoroptic probes inserted into the superficial dermis and subcutaneous fat of an anesthetized pig. RF energy treatments (200 J/3 cm² or 300 J/3 cm² over 8 seconds) were applied to the skin over the temperature probe insertion areas. Baseline and treatment temperatures were monitored in real time in order to permit the tissue to cool between RF energy pulses. With RF energy treatment (300 J/3 cm²), the temperature of the subcutaneous adipose tissue increased from approximately 35° C. to 55° C. (FIG. 3). The dermis temperature remained relatively cool and increased only from 33° C. to 36° C. in response to the RF energy treatment. The lower treatment setting (200 J/3 cm² over 8 seconds) produced a moderate subcutaneous temperature increase from a baseline temperature of 35° C. to peak temperatures of 42° C. to 49° C. The observation that the subcutaneous tissue was selectively heated was reproducible from day to day, as shown in FIG. 4.

In addition, a stacked treatment protocol of 255 J/3 cm² applied over 8 second pulses was applied to the pig skin. A stacked pulse does not allow the tissue to cool to baseline temperatures between consecutive RF energy pulses. In this instance, the subcutaneous temperatures increased from a baseline temperature of 35° C. to a temperature of 78° C., a relatively extreme temperature well above therapeutic treatment temperatures, while the temperature of the underlying muscle tissue increased modestly from 37° C. to 43° C. (FIG. 5).

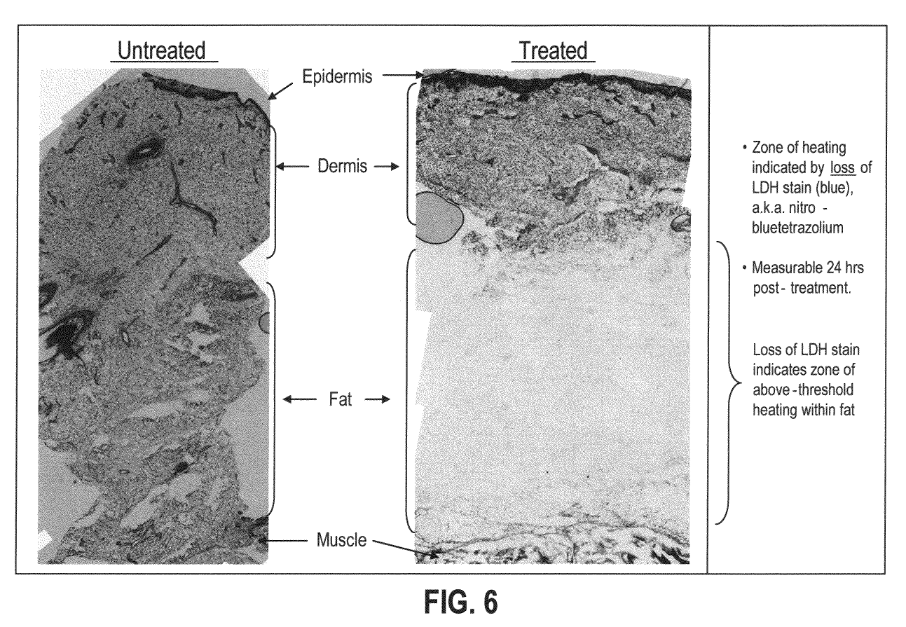

Histological evaluation was conducted to look at indicators of acute thermal damage as well as long-term processes resulting from thermal damage. Thermal damage to the subcutis was observed by nitroblue-tetrazolium chloride staining (NBTC) otherwise referred to as lactate dehydrogenase (LDH) staining. LDH staining is a method to assess the presence of LDH enzyme activity in a tissue slice. Thermal treatments that raise the temperature of adipose tissue to the threshold temperature to about 55° C. to about 59° C. results in loss of LDH enzyme activity. Untreated tissue, when subjected to LDH staining, exhibits a relatively uniform blue stain throughout the tissue layers indicating a basal level of LDH activity. Heating a tissue to above the threshold results in loss of staining, and thus a cleared zone is observed in the tissue section. Twenty-four hours after RF energy application of 400 J/3 cm² for 9.6 seconds to live pig skin, a loss of LDH staining in the subcutaneous fat layer was observed, suggesting that this layer was heated to above the threshold level (FIG. 6). In contrast, the dermis was stained blue indicating that the RF energy application had no effect on LDH activity (FIG. 6).

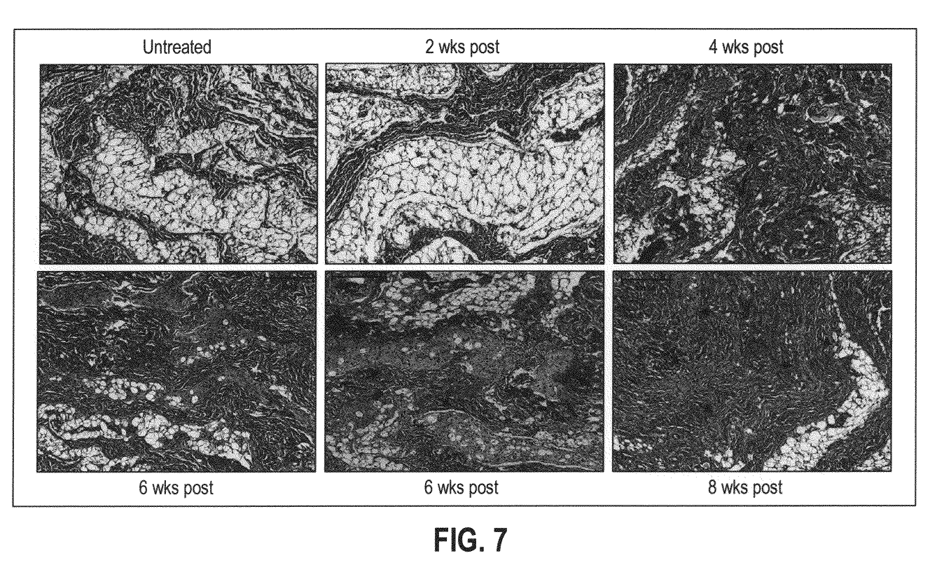

Tissue responses of live pig skin were evaluated up to 8 weeks post-treatment using histological methods. As seen in FIG. 7, a low grade inflammatory response and adipose tissue necrosis was observed between 2 and 8 weeks post-treatment as evidenced by the presence of foamy macrophages and occasional giant cells within the affected fat lobules, as indicated by arrows. In addition, fat loss was observed within the treated tissue as evidence by fibroplasia in areas of reduced fat lobules. Other associated inflammatory responses, such as lymphocytic infiltration were generally mild. Adipose tissue necrosis was ongoing at 8 weeks post-treatment.

The results from the pig studies were extended to human studies in which abdominal tissue was treated on subjects at various time intervals prior to an abdominoplasty procedure. At the time of abdominoplasty, the treated tissue was excised and processed for histological evaluation.

RF energy treatments were performed with the concept device and modified treatment protocol of low RF energies applied over a longer period of time to the abdomen skin of subjects who were scheduled for a future abdominoplasty. No topical or other anesthetics were used. Subjects were treated at settings that generated a very warm or hot sensation with tolerable or no discomfort. Abdominoplasty surgeries occurred after 1, 2, or 3 months post-treatment.

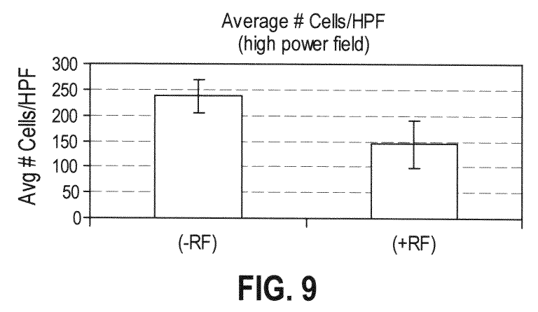

Skin tissues that were treated with the above concept device and protocols were evaluated for necrosis, macrophage infiltration (CD68), adipocyte lipid droplet protein perilipin, fibroblast marker protein HSP47, and new collagen deposition (trichrome). Histological changes consistent with adipose tissue necrosis or fat cell death (macrophages surrounding adipocytes) were observed between 4 mm and 23 mm within the subcutaneous adipose tissue layer as indicated by arrows in the center and right panels of FIG. 8. The left panel of FIG. 8 demonstrates that there is no histological evidence of tissue necrosis or fat cell death in untreated tissue. Fibroblast presence or mild fibrosis (new collagen deposition and fibroplasia) was observed at 2 and 3 months post-treatment which is consistent with tissue remodeling after thermal injury. In the necrotic area, the number of fat cells was decreased as determined by morphometric analysis of histological sections (FIG. 9).

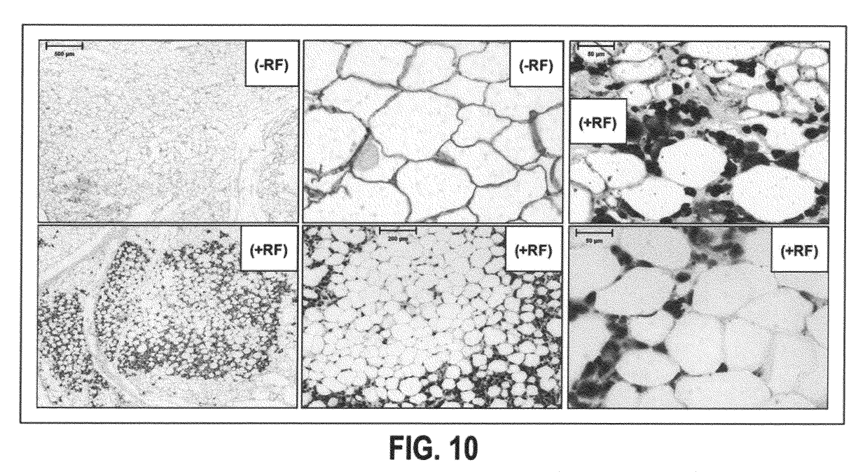

Perilipin protein was observed to be absent in certain treatment areas that did or did not exhibit evidence of necrosis (FIG. 10). Perilipin is a protein that protects the lipid droplet within the fat cell from enzyme-mediated lipid hydrolysis. Loss of perilipin protein has been shown to indicate fat cell death where macrophages are also present, or an increase in fat cell lipolytic activity in instances where macrophages are not present (Strissel et al., (2007) Diabetes, 56:2910; Cinti et al., (2005) J. Lipid Res., 46:2347; Tansey et al., (2001) PNAS, 98:6494; Kovsan et al., (2007) J Biol. Chem., 282:21704; Kern et al., (2004) J. Clin. Endocrinol. Metab., 89:1352; Mottagui-Tabar et al., (2003) Diabetologia, 46: 789; Martinez-Botas et al., (2000) Nat. Genet., 26:474; and Qi et al., (2004) Obes. Res., 12:1758, each of which are hereby incorporated by reference herein in their entirety). FIG. 10 demonstrates changes in perilipin expression and macrophage infiltration that results from treatment of tissue using the methods and devices described herein. Specifically, perilipin, indicated by blue staining, is present around adipocyte membranes in untreated tissue (upper left and upper center). Macrophages are present, as indicated by brown CD68 staining, in necrotic fat lobules surrounding dead fat cells (as indicated by arrows in upper right, lower left, lower center, and lower right panels). Perilipin staining was also absent in treated tissue even in the absence of necrosis (as indicated by arrow heads in lower center and lower right panels). The observed loss of perilipin in adipose tissue treated with RF energy suggests that RF energy may affect fat cell metabolic activity resulting in decreased fat accumulation. Thus, RF energy may reduce adipose tissue fat content by decreasing the number of fat cells or by decreasing perilipin and thereby increasing lipid hydrolysis.

While the invention has been illustrated by a description of various embodiments and while these embodiments have been described in considerable detail, it is not the intention of the applicant to restrict or in any way limit the scope of the appended claims to such detail. For example, it is understood that the present methodologies could be utilized to cause selective heating of any tissue having a higher fat content than surrounding tissues and is not limited to subcutaneous adipose tissue. Additional advantages and modifications will readily appear to those skilled in the art. Thus, the invention in its broader aspects is therefore not limited to the specific details, representative apparatus and method, and illustrative example shown and described. Accordingly, departures may be made from such details without departing from the spirit or scope of applicant's general inventive concept.

What is claimed is:

1. A method of operating a tissue treatment apparatus for heating adipose tissue located beneath the dermis, the method comprising:
delivering high frequency energy from an electrode to the adipose tissue at a dose and for a period of time that are sufficient to heat the adipose tissue to at least 42° C. with the proviso that the temperature of the dermis is about 42° C. or less, wherein the dose is in a range of about 3 W/cm² to about 100 W/cm², and the period of time ranges from greater than 3 seconds to about 30 seconds.

2. The method of claim 1 further comprising:
cooling the dermis by heat transfer from the skin to the electrode in order to maintain the temperature of the dermis at 42° C. or less.

3. The method of claim 1 wherein the temperature of the dermis is about 40° C. or less.

4. The method of claim 1 wherein the dose is in a range of about 9 J/cm² to about 800 J/cm².

5. The method of claim 1 wherein the dose is sufficient to heat the adipose tissue to 45° C. or higher.

6. The method of claim 1 wherein the high frequency energy is applied in one of at least a first pulse or a plurality of pulses.

7. The method of claim 6 wherein the tissue is allowed to return to its pre-treatment temperature after an initial pulse from among the plurality of pulses.

8. The method of claim 6 wherein the tissue is not allowed to return to its pre-treatment temperature prior to the application of the additional pulse.

9. The method of claim 1 further comprising:
measuring the temperature of at least one of the adipose tissue or the dermis.

10. A method of operating a tissue treatment apparatus for heating adipose tissue located beneath the dermis, the method comprising:
delivering high frequency energy from an electrode to the adipose tissue at a dose and for a period of time that are sufficient to increase at least one of necrosis, apoptosis, or inflammation in the treated adipose tissue relative to an untreated adipose tissue, wherein the dose is in a range of about 3 W/cm² to about 100 W/cm², and the period of time ranges from greater than 3 seconds to about 30 seconds.

11. The method of claim 10 further comprising:
cooling the dermis to maintain the temperature of the dermis at 42° C. or less.

12. The method of claim 10 further comprising:
measuring the increase of at least one of necrosis, apoptosis, or inflammation using a histological staining method.

13. The method of claim 10 wherein the dose is in a range of about 9 J/cm² to about 800 J/cm².

14. A method of operating a tissue treatment apparatus for heating adipose tissue located beneath the dermis, the method comprising:
delivering high frequency energy from an electrode to the adipose tissue at a dose and for a period of time that are sufficient to decrease perilipin activity, wherein the dose is in a range of about 3 W/cm² to about 100 W/cm², and the period of time ranges from greater than 3 seconds to about 30 seconds.

15. The method of claim 14 further comprising:
cooling the dermis to maintain the temperature of the dermis at 42° C. or less.

16. The method of claim 14 wherein perilipin activity is decreased by decreasing expression of at least one of a perilipin gene or protein.

17. The method of claim 14 wherein the dose is in a range of about 8 J/cm² to about 200 J/cm².
